(19) United States
(12) Patent Application Publication
(10) Pub. No.: US 2015/0065442 A1
(43) Pub. Date: Mar. 5, 2015

(54) REDUCIBLE SELF-ASSEMBLED MICELLE DRUG DELIVERY SYSTEMS

(71) Applicant: University of Manitoba, Winnipeg (CA)

(72) Inventor: Malcolm M.Q. Xing, Winnipeg (CA)

(21) Appl. No.: 14/372,334

(22) PCT Filed: Jan. 15, 2013
(86) PCT No.: PCT/CA2013/000039

§ 371 Date: Jul. 15, 2014

Related U.S. Application Data
(60) Provisional application No. 61/586,833, filed on Jan. 15, 2012.

(51) Int. Cl. A61K 47/34; A61K 31/704

(52) U.S. Cl. CPC A61K 47/34; A61K 31/704
USPC 514/34; 514/772.3

(57) ABSTRACT

Reductively degradable micelles comprising poly(-βamino ester)s-g-poly(ethylene glycol) amphiphilic copolymers provided with aromatic phenylbutylamine side groups, suitable for sequestering therein anthrocyclines. The anthrocycline-loaded reductively degradable micelles are useful for therapeutic treatment of cancers.

### FIELD OF THE INVENTION

The present invention pertains to compositions and systems for intracellar delivery of therapeutic agents, and more particularly, self-assembled co-polymeric micelle systems for intracellar delivery of therapeutic organic molecules and/or inorganic molecules.

### BACKGROUND OF THE INVENTION

Anthracyclines are a class of antibiotics derived from Streptomyces bacteria. While effective toward the inhibition of bacteria growth, their potent cytotoxicity towards mammallian cells has hindered the clinical use of these compounds to treat infections. However, anthracyclines have found widespread use as anticancer agents. There are three mechanims of action by which this class of compounds is thought to act as antiproliferative agents: DNA intercalation, Topoisomerase II inhibition, and free radical production to induce DNA damage. Due to the multiple mechanisms of action, anthracyclines are toxic against a broad spectrum of cell lines, and thus effective against multiple types of cancer.

Several anthracycline derivatives have been produced and have found use in the clinic for the treatment of leukemias, Hodgkin's lymphoma, as well as cancers of the bladder, breast, stomach, lung, ovaries, thyroid, and soft tissue sarcoma. Such anthracycline derivatives include daunorubicin, doxorubicin, epirubicin, idarubicin, and valrubicin. Anthracyclines are typically prepared as an ammonium salt (e.g. hydrochloride salt) to improve water solubility and allow for case of administration.

In particular, doxorubicin (DOX) is a clinically prevalent anticancer drug that has been widely used in the treatment of different types of tumors. The kinetics of DOX is known to interact with DNA by intercalation and to inhibit the biosynthesis of macromolecules. It is crucial, therefore, to deliver DOX into the cytoplasm and/or the cell nucleus. Meanwhile, severe, dose-limiting side-effects such as hypersensitivity and cardiotoxicity of doxorubicin impede its clinical application. Therefore, there is an urgent need for developing safer and more effective DOX delivery systems.

Polymeric micelles have been studied extensively and well-established as nano-scaled drug carriers for regulated release of various hydrophobic anticancer drugs including DOX and paclitaxel, which were self-assembled into micelles from amphiphilic graft or block copolymers. The core-shell structure of polymeric micelles is essential in their pharmaceutical applications. The hydrophobic core is usually loaded with a variety of therapeutic or diagnostic agents, while the hydrophilic shell, like a corona, stabilizes the micelles in an aqueous solution. Poly(ethylene glycol) (PEG) is one of the most widely used hydrophilic moieties, because it is highly hydrated, readily water-soluble, non-toxic and non-immunogenic. Therefore, PEG chains can efficiently prevent the interactions of the micelles with serum proteins and cells, avoid particle opsonization, and render them “unrecognizable” by the reticuloendothelial system (RES) in the liver and spleen. Also, the nano-scale of the micelles allows them to escape rapid clearance by the RES.

In principle, escape from clearance enhances the opportunity for accumulation in tumors where the vessel wall increases its pore size. The use of copolymers that are sensitive to temperature, pH and reduction potential can provide a stimuli-responsiveness for controlled drug release. There is about a 1,000× difference of reductant concentration between the extracellular environment (micromolar) and the various subcellular organelles in cytoplasm (millimolar). Disulfide bonds in the structure of nanoparticle are reductively degraded in the reducing intracellular environment easily, while remaining in a predominantly oxidizing extracellular space. The intracellular cleavage of disulfide bonds in nanoparticles is mostly mediated by thiol/disulfide exchange reactions with small redox molecules such as glutathione (GSH), either alone or with the help of redox enzymes.

Reductively degradable micelles or micelles with reductive cleavable shell have been reported to improve intracellular drug release. However, complicated synthesis techniques are needed to obtain those copolymers, and the role of disulfide bonds in those drug delivery systems needs more comprehensive understanding.

### SUMMARY OF THE INVENTION

The exemplary embodiments of the present disclosure pertain to reductively degradable self-assembled micelles comprising of poly(β-amino ester)-graft-poly(ethylene glycol) amphiphilic copolymers with phenylbutylamine functional side groups. The reductively degradable self-assembled micelles are useful for sequestering therein anthrocyclines exemplified by doxorubicin. The aromatic phenylbutylamine side groups increase hydrophobicity and strengthen the interaction with anthrocyclines to improve the micelles'drug-loading capability and capacity. Exemplary methods for producing the reductively degradable self-assembled micelles are disclosed.

Some exemplary embodiments pertain to anthrocyclin-loaded reductively degradable self-assembled micelles. Exemplary methods for producing anthrocycline-loaded reductively degradable self-assembled micelles are disclosed.

### BRIEF DESCRIPTION OF THE DRAWINGS

The present invention will be described in conjunction with reference to the following drawings in which:

FIG. 1 is a schematic diagram of an exemplary pathway for preparing the reducible self-assembled micelles of the present invention;

FIG. 2(A) is a chart showing the typical size and size distribution of exemplary reducible self-assembled micelles of the present invention, and 2(B) is a transmission electron microscopy micrograph of the exemplary reducible self-assembled micelles of the present invention;

FIG. 3 is a chart showing the effects of dithiotreitol on the sizes of the exemplary reducible self-assembled micelles;

FIG. 4 is a chart showing the effects of dithiotreitol on the disassembly of the exemplary reducible self-assembled micelles;

FIG. 5 is a chart showing the effects of dithiotreitol on the release of doxorubicin (DOX) from the exemplary reducible self-assembled micelles at 37° C.;

FIG. 6 is a chart showing inhibition of HepG2 liver cancer cells by different concentrations of DOX delivered by the exemplary reducible self-assembled PAE-1 micelles or PAE-4 micells;

FIG. 7 is a chart showing inhibition of HepG2 liver cancer cells after 24 h and 48 h of exposure incubation with DOX-loaded reducible self-assembled PAE-1 micelles or PAE-4 micells;

FIGS. 8(A-D) are micrographs of HepG2 liver cancer cells after: (A) and (B) a 15-min incubation with free DOX; (C) and (D) a 15-min incubation with DOX-loaded reducible self-assembled PAE-1 micelles. Images (A) and (C) were produced by overly of cells with DOX fluorescence, nuclear staining with Topro-3 and F-actin staining with phallacidin; while images (B) and (D) were produced with DOX fluorescence only;

FIGS. 9(A-D) are micrographs of HepG2 liver cancer cells after: (A) and (B) a 2-h incubation with free DOX; (C) and (D) a 2-h incubation with DOX-loaded reducible self-assembled PAE-1 micelles. Images (A) and (C) were produced by overly of cells with DOX fluorescence, nuclear staining with Topro-3 and F-actin staining with phallacidin; while images (B) and (D) were produced with DOX fluorescence only;

FIGS. 10(A-D) are micrographs of HepG2 liver cancer cells after: (A) and (B) a 24-h incubation with free DOX; (C) and (D) a 24-h incubation with DOX-loaded reducible self-assembled PAE-1 micelles. Images (A) and (C) were produced by overly of cells with DOX fluorescence, nuclear staining with Topro-3 and F-actin staining with phallacidin; while images (B) and (D) were produced with DOX fluorescence only;

FIGS. 11(A-D) are micrographs of MCF7 human breast cancer cells after: (A) and (B) a 15-min incubation with free DOX; (C) and (D) a 2-h incubation with free DOX. Images (A) and (C) were produced by overly of cells with DOX fluorescence, nuclear staining with Topro-3 and F-actin staining with phallacidin, while images (B) and (D) were produced with DOX fluorescence only;

FIGS. 12(A-D) are micrographs of MCF7 human breast cancer cells after: (A) and (B) a 15-min incubation with DOX-loaded reducible self-assembled rPAE micelles; (C) and (D) a 2-h incubation with DOX-loaded reducible self-assembled rPAE micelles. Images (A) and (C) were produced by overly of cells with DOX fluorescence, nuclear staining with Topro-3 and F-actin staining with phallacidin, while images (B) and (D) were produced with DOX fluorescence only; and

FIGS. 13(A-D) are micrographs of MCF7 human breast cancer cells after: (A) and (B) a 15-min incubation with DOX-loaded non-reducible self-assembled nPAE micelles; (C) and (D) a 2-h incubation with DOX-loaded non-reducible self-assembled nPAE micelles. Images (A) and (C) were produced by overly of cells with DOX fluorescence, nuclear staining with Topro-3 and F-actin staining with phallacidin, while images (B) and (D) were produced with DOX fluorescence only.

### DESCRIPTION OF THE INVENTION

Unless otherwise defined, all technical and scientific terms used herein have the same meaning as commonly understood by one of ordinary skill in the art to which this invention belongs. In order that the invention herein described may be fully understood, the following terms and definitions are provided herein.

The word “comprise” or variations such as “comprises” or “comprising” will be understood to imply the inclusion of a stated integer or groups of integers but not the exclusion of any other integer or group of integers.

The term “about” or “approximately” means within 20%, preferably within 10%, and more preferably within 5% of a given value or range.

The term “subject” means an animal, preferably a mammal, and more preferably a human.

The term “multiblock copolymer” refers to a polymer comprising one synthetic polymer portion and two or more poly(amino acid) portions. Such multiblock copolymers include those having the format W—X′—X″, wherein W is a synthetic polymer portion and X and X′ are poly(amino acid) chains or “amino acid blocks”. In certain embodiments, the multiblock copolymers of the present invention are triblock copolymers. As described herein, one or more of the amino acid blocks may be “mixed blocks”, meaning that these blocks can contain a mixture of amino acid monomers thereby creating multiblock copolymers of the present invention. In some embodiments, the multiblock copolymers of the present invention comprise a mixed amino acid block and are tetrablock copolymers.

The term “portion” or “block” refers to a repeating polymeric sequence of defined composition. A portion or a block may consist of a single monomer or may be comprise of one or more monomers, resulting in a “mixed block”. One skilled in the art will recognize that a monomer repeat unit is defined by parentheses around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by (A)₄(B)₄(C)₄(D)₄].

The term “tri-block copolymer” refers to a polymer comprising one synthetic polymer portion and two poly(amino acid) portions.

The term “inner core” as it applies to a micelle of the present invention refers to the center of the micelle formed by the hydrophobic poly(amino acid) block. In accordance with the present invention, the inner core is not cross-linked. By way of illustration, in a tri-block polymer of the format W—X′—X″, as described above, the inner core corresponds to the X″ block.

The term “outer core” as it applies to a micelle of the present invention refers to the layer formed by the first poly(amino acid) block. The outer core lies between the inner core and the hydrophilic shell. In accordance with the present invention, the outer core is either crosslinkable or is cross-linked. By way of illustration, in a triblock polymer of the format W—X′—X″, as described above, the outer core corresponds to the X′ block. It is contemplated that the X′ block can be a mixed block.

The terms “drug-loaded” and “encapsulated”, and derivatives thereof, are used interchangeably. In accordance with the present invention, a “drug-loaded” micelle refers to a micelle having a drug, or therapeutic agent, situated within the core of the micelle. In certain instances, the drug or therapeuctic agent is situated at the interface between the core and the hydrophilic coronoa. This is also referred to as a drug, or therapeutic agent, being “encapsulated” within the micelle.

The term “polymeric hydrophilic block” refers to a polymer that is not a poly(amino acid) and is hydrophilic in nature. Such hydrophilic polymers are well known in the art and include polyethyleneoxide (also referred to as polyethylene glycol or PEG), and derivatives thereof, poly(N-vinyl-2-pyrolidone), and derivatives thereof, poly(N-isopropylacrylamide), and derivatives thereof, poly(hydroxyethyl acrylate), and derivatives thereof, poly(hydroxylethyl methacrylate), and derivatives thereof, and polymers of N-Q-hydroxypropoyl)methacrylamide (HMPA) and derivatives thereof.

The term “poly(amino acid)” or “amino acid block” refers to a covalently linked amino acid chain wherein each monomer is an amino acid unit. Such amino acid units include natural and unnatural amino acids. In certain embodiments, each amino acid unit of the optionally a crosslinkable or crosslinked poly(amino acid block) is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, i.e., blocks comprising a mixture of amino acid residues.

The term “D,L-mixed poly(amino acid) block” refers to a poly(amino acid) block wherein the poly(amino acid) consists of a mixture of amino acids in both the D- and L-configurations. In certain embodiments, the D,L-mixed poly(amino acid) block is hydrophobic. In other embodiments, the D,L-mixed poly(amino acid) block consists of a mixture of D-configured hydrophobic amino acids and L-configured hydrophilic amino acid side-chain groups such that the overall poly(amino acid) block comprising is hydrophobic.

Exemplary poly(amino acids) include poly(benzyl glutamate), poly(benzyl aspartate), poly(L-leucine-co-tyrosine), poly(D-leucine-co-tyrosine), poly(L-phenylalanine-co-tyrosine), poly(D-phenylalanine-co-tyrosine), poly(L-leucine-coaspartic acid), poly(D-leucine-co-aspartic acid), poly(L-phenylalanine-co-aspartic acid), poly(D-phenylalanine-co-aspartic acid).

The term “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

The phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

The term “tacticity” refers to the stereochemistry of the poly(amino acid) hydrophobic block. A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer.

The term anthracycline refers to a class of antibiotics derived from Streptomyces bacteria. Exemplary anthracyclines include, but are not limited to, daunorubicin, doxorubicin, epirubicin, idarubicin, valrubicin, and salts thereof.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as in neutron scattering experiments, as analytical tools or probes in biological assays.

The term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g., Fe₃O₄ and Fe₂O₃) particles. Similarly, semiconducting nanoparticles (e.g., cadmium selenide, cadmium sulfide, cadmium telluride) are useful as fluorescent labels. Other metal nanoparticles (e.g colloidal gold) also serve as primary labels.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength.

The term “pharmaceutically acceptable carrier, adjuvant, or vehicle” refers to a nontoxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxy ethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

The term “micelle” means an aggregate of spherical surfactant molecules dispersed in a liquid colloid wherein the aggregate comprises outward-facing hydrophilic head regions sequestering hydrophobic single tail regions in the micelle centre.

The acronym “PAEs” refers to micelles that are approximately 100 nm in diameter and consist of poly(β-amino ester)-graft-poly(ethylene glycol) amphiphilic copolymers (PAE) with phenylbutylamine functional side groups.

The exemplary embodiments of the present disclosure pertain to reductively degradable micelles comprising poly(β-amino ester)s-g-poly(ethylene glycol) amphiphilic copolymers provided with aromatic phenylbutylamine side groups, and are referred to herein as “rPAEs”. A suitable aromatic phenylbutylamine side group is exemplified by butylbenzene. The degree of hydrophobicity of the present rPAE micelles can be tailored and adjusted by use of different chain lengths of the aromatic phenylbutylamines.

The rPAE micelles disclosed herein are particularly suitable for sequestering anthracycline derivatives, and can be specifically prepared for a selected anthracycline derivative for example, for doxorubicin (DOX), by selecting a certain chain-length of butylbenzene.

The exemplary rPAE micelles of the present disclosure can be produced by simple and mild methods based on the one-step Michael addition polymerization process to synthesize a series of novel disulfide bonds that contain poly(β-amino ester)s (PAE) with poly(ethylene glycol)s and butylbenzene as side chains.

The rPAE micelles sequestering anthracyclines as disclosed herein are particularly useful for intracellular delivery of the anthracycline derivatives to and into cancer cells, cancerous growths, and cancer tumors. Accordingly, anthracycline-loaded rPAE micelles of the present disclosure are useful for treatment of liver cancer, breast cancer, prostrate cancer, colorectal cancer, pancreatic cancer, a cancer of the ovary, cervix, testis, genitourinary tract, esophagus, larynx, glioblasts, neuroblasts, stomach, skin, lungs, bone, colon, bladder, skin, kidney, lip, tongue, mouth, pharynx, small intestine, large intestine, rectum, brain and central nervous system.

Accordingly, another embodiment of the present disclosure relates to compositions comprising a rPAE micelle loaded with an anthracycline or a pharmaceutically acceptable derivative thereof and a pharmaceutically acceptable carrier, adjuvant, or vehicle. The compositions of this disclosure are formulated for administration to a subject in need of such composition, and are administered orally, parenterally, by inhalation spray, topically, rectally, nasally, buccally, vaginally or via an implanted reservoir. The term “parenteral” as used herein includes subcutaneous, intravenous, intramuscular, intraarticular, intra-synovial, intrasternal, intrathecal, intrahepatic, intralesional and intracranial injection or infusion techniques.

Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the drug can be administered to a patient receiving these compositions.

It will be appreciated that dosages typically employed for the encapsulated drug are contemplated by the present invention. In certain embodiments, a patient is administered a drug-loaded micelle of the present invention wherein the dosage of the drug is equivalent to what is typically administered for that drug. In other embodiments, a patient is administered a drug-loaded micelle of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The disclosure includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

In order that the disclosure disclosed herein may be more fully understood, the following examples are set forth. It will be understood that these examples are for illustrative purposes only and are not to be construed as limiting this disclosure in any manner.

### EXAMPLES

The following materials and instruments were used in the examples disclosed herein.

### Materials:

All chemicals were purchased from Aldrich Chemical Co. (St. Louis, Mo., US) and used without further purification unless otherwise noted. Methoxy PEG (5K) and Methoxy PEG(5K)—NH₂ (mPEG-NH₂) were purchased from JenKem Technology USA Inc. (Allen, Tex., US). Dialysis membrane (7 kDa MWCO), 0.45 μm Millipore® Millex® syringe filters (Millipore and Millex are registered trademarks of Merck KGAA, Darmstadt, Fed. Rep. Germany) were purchased from Fisher Scientific (Ottawa, ON, Canada). Carbon-coated copper grids were purchased from Canemco Inc. (Core, PQ, Canada). HepG2 was purchased from ATCC (Manassas, Va., USA). MCF-7 cells were a gift from Dr. A. Raouf (Manitoba Cancer Care, Winnipeg, MB, Canada).

### Instruments:

¹H-NMR spectra were recorded using a Bruker Avance 300 NMR spectrometer (300 MHz) with CDCl₃ as the solvent. The UV absorbance was recorded with a Varian Cary-50 UV-vis spectrophotometer. The fluorescence intensity of pyrene was recorded with a Varian Cary Eclipse fluorescence spectrophotometer.

The number average (Mn), weight average (Mw) molecular weight and polydispersity index (PDI) Mw/Mn were determined by a size exclusion chromatography (SEC) system consisting of Shimadzu LC-10ADVP solvent delivery unit, a CTO-10ASVP Shimadzu column oven and a Polymer Laboratories PL-gel 5 μm mixed C column. The system was also equipped with a mini DAWN triangle light scattering detector and an OPTILAB DSP interferometric refractometer (both distributed by Wyatt Technology, Santa Barbara, Calif., USA). THF was used as eluent at a flow rate of 1.0 ml/min and temperature of 30° C. SEC data were analyzed using Astra software from Wyatt Technology. Refractive index increments (dn/dc) of PAEs were determined by an interferometric refractometer and used in the SEC analysis.

Images of the micelles were recorded by a Joel 1010 Transmission Electron Microscope (TEM) at 80 KV using a LaB6 filament and recorded using an AMT digital camera. Micrographs were collected at 60,000× magnification. Hydrodynamic diameters (Dh) and size distributions were determined by Malvern Zetasizer Nano-S dynamic light scattering (DLS) (Malvern Instruments Ltd. Worcestershire, UK). Measurements were conducted at room temperature.

### Example 1

### Preparation and Characterization of the Reducible Self-Assembled PAE Micelles

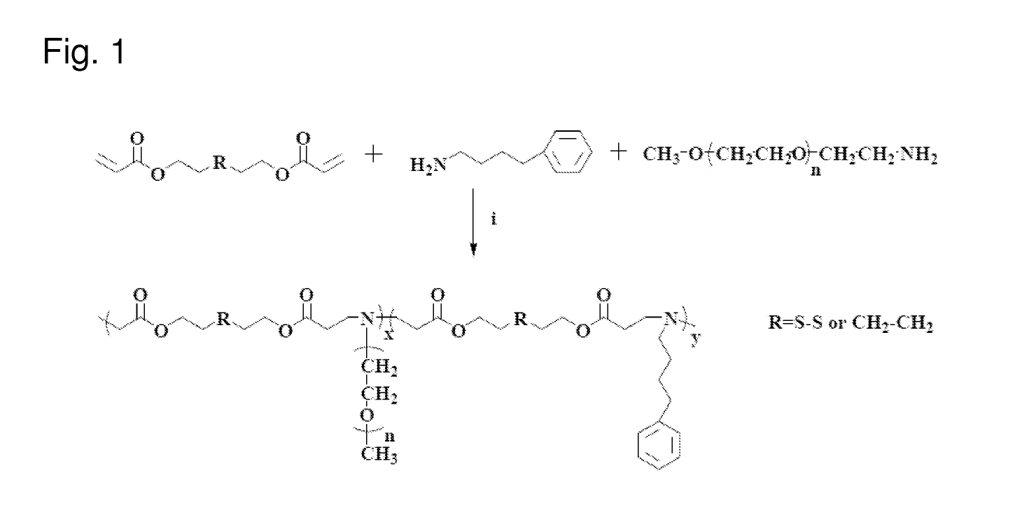

The reductive degradable PAEs graft copolymers were synthesized via a one-step Michael addition polymerization as shown in FIG. 1.

First, the 2,2′-dithiodiethanol diacrylate (DTDA) was synthesized as taught by Hong et al., (2007, Thermal control over the topology of cleavable polymers: From linear to hyperbranched structures. J. Am. Chem. Soc. 129:5354-5355) and Chen et al. (2011, pH and Reduction Dual-Sensitive Copolymeric Micelles for Intracellular Doxorubicin Delivery. Biomacromolecules 12:3601-3611).

Next, the DTDA was allowed to react with primary amines via the Michael addition to form the poly(β-amino ester)s, which contained the disulfide linkage in the backbones, as follows. DTDA (262 mg, 1.0 mmol), 4-phenylbutylamine (134 mg, 0.9 mmol) and mPEG-NH2 (500 mg, 0.1 mmol) were added into a 20-ml borosilicate vial containing a magnetic stirrer bar, and 4 ml DMSO was added while stirring to dissolve the mixture to a clear solution. The reaction proceeded 65° C. for about 72 h. The mixture was precipitated in a large amount of diethyl ether three times, and dried in under vacuum for two days. The typical yield of the copolymer was about 80%.

To make amphiphilic poly(β-amino ester)s, we adopted phenylbutylamine and mPEG-amine functional side groups. Using different proportions of two kinds of acrylate monomers and two primary amine-monomers, two types of poly(β-amino ester)s were produced (Table 1), one with disulfide linkages (i.e., reducible PAE or rPAE) and the second without disulfide linkages (i.e., non-reducible PAE or nPAE).

Reducible PAE (rPAE) copolymer ¹H-NMR (ppm): δ 1.40-1.60 (4H, —(CH₂)₂CH₂-benzene), 2.50-2.60 (4H, —CH₂—COO— and 2H, —(CH₂)₂CH₂-benzene and 2H, PEG-CH₂—CH₂—N), 2.80-3.00 (4H, —(S—CH₂)₂— and 4H, —N(CH₂)₂—), 3.50-3.70 (CH₂ in PEG repeat unit), 4.30-4.40 (4H, —COOCH₂—), 7.20-7.40 (5H, CH in benzene).

Non-reducible PAE (nPAE) copolymer ¹H-NMR (ppm): δ 1.30-1.40 (4H, —CH₂(CH₂)₂CH₂—), δ 1.40-1.60 (4H, —(CH₂)₂CH₂-benzene and 4H, —CH₂(CH₂)₂CH₂—), 2.50-2.60 (4H, —CH₂—COO— and 2H, —(CH₂)₂CH₂-benzene and 2H, PEG-CH₂—CH₂—N), 2.80-3.00 (4H, —N(CH₂)₂—), 3.50-3.70 (CH₂ in PEG repeat unit), 4.05-4.20 (4H, —COOCH₂—), 7.20-7.40 (5H, CH in benzene).

TABLE 1
Characterization of chemical structure of the PAE copolymers.

| | Disulfide | | | | | | |
|---|---|---|---|---|---|---|---|
| | linkage in | Benzene/PEG | | | | | |
| | backbone | (mol) | | Yield | Mn^{b} | Mw^{b} | |
| Sample | (mol %) | Feed | Polymer^{a} | (%) | (KDa) | (KDa) | PDI^{b} |
| rPAE | 100 | 9/1 | 7/1 | 74.3 | 14.0 | 18.9 | 1.35 |
| nPAE | 0 | 9/1 | 7/1 | 67.2 | 23.8 | 27.6 | 1.16 |

^{a}Defined by ¹H-NMR
^{b}Determined by GPC-light scattering combined system.

¹H-NMR results indicated that structures of the PAE were slightly different from the predicted structures, which may have been caused by the different reactivity of amines in phenylbutylamine and mPEG-amine. Elevated reaction temperature was also employed to increase reactivity of secondary amines (formed) to yield tertiary amine groups that formed grafting structure. The amphiphilic copolymers were purified by precipitation in an excess amount of ether (two times). The GPC light scattering method was used to determine the molecular weight and the polydispersity of the copolymers, which demonstrated successful preparation of monodial copolymer with narrow molecular weight distribution (Table 1).

The critical micellation concentration (CMC) of PAE copolymer in water was estimated by fluorescence spectroscopy using pyrene as a probe. Twenty μl of pyrene acetone solution (20 μg/ml) were added to 4 ml vials, and then acetone was allowed to evaporate. Four ml of aqueous solution containing 0.1-128 mg/l of PAE copolymers were added to the vials. The final concentration of pyrene in each sample solution was 0.1 μg/ml. The excitation spectra (300-360 nm) of the solutions were recorded at an emission wavelength set at 395 mm and slits adjusted to give a bandpass of 5 nm for excitation and emission beams. The ratios of the peak intensities at 338 nm over 334 nm (I338/I334) of the excitation spectra were recorded and plotted versus polymer concentration. The CMC value was taken from the intersection points of the tangent to the curve at the high concentrations with the horizontal line through the point at the low concentrations. The CMC values of the copolymers are listed in Table 2. The CMC of PAE was in the range of 25-30 mg/l, indicating that a stable core-shell structure remained in a low concentration.

TABLE 2
Characterization of PAE polymeric micelles and DOX-loaded PAE
polymeric micelles.

| | Micelle | | | | | | Drug |
|---|---|---|---|---|---|---|---|
| | diameter^{a} | | | | | Drug | loading |
| | (nm) | | Polydispersity^{a} | | | loading | effi- |
| Sam- | DOX- | DOX- | DOX- | DOX- | CMC^{b} | content^{c} | ciency^{c} |
| ple | free | loaded | free | loaded | (mg/l) | (%) | (%) |
| rPAE | 114.7 | 118.5 | 0.103 | 0.105 | 25.1 | 2.72 | 56 |
| nPAE | 127.2 | 133.8 | 0.093 | 0.095 | 28.2 | 3.05 | 63 |

^{a}Size and PDI of micelles and DOX-loaded micelles were determined by DLS.
^{b}Determined by fluorescence spectrometry using pyrene as probe.
^{c}Determined by UV-vis absorbance measurement.

Hydrodynamic diameter and size distribution of the micelles were determined by dynamic light scattering (DLS). DLS measurements were carried out at 20° C. using Zetasizer Nano-S from Malvern Instruments. Solution of the micelles (250 mg/l) was passed through a 0.45-μm pore size filter prior to being measured. The morphology of micelles was examined by transmission electron microscopy (TEM). The digital images were taken by a Joel 1010 TEM at 60 KV using a LaB6 filament and recorded using an AMT digital camera. The TEM samples were prepared as As follows. A drop of the isolated micelle solution was drop-cast on a carbon-coated copper grid (400-mesh) and dried by filter paper. Then, a 2% (w/v) of uranyl acetate solution was dropped on the grid. One minute of contact was allowed before excess liquid was removed using filter paper. The grid was air-dried for 1 h before being observed under a microscope. Then the average diameter of particles was measured from 10 particles in the TEM micrographs.

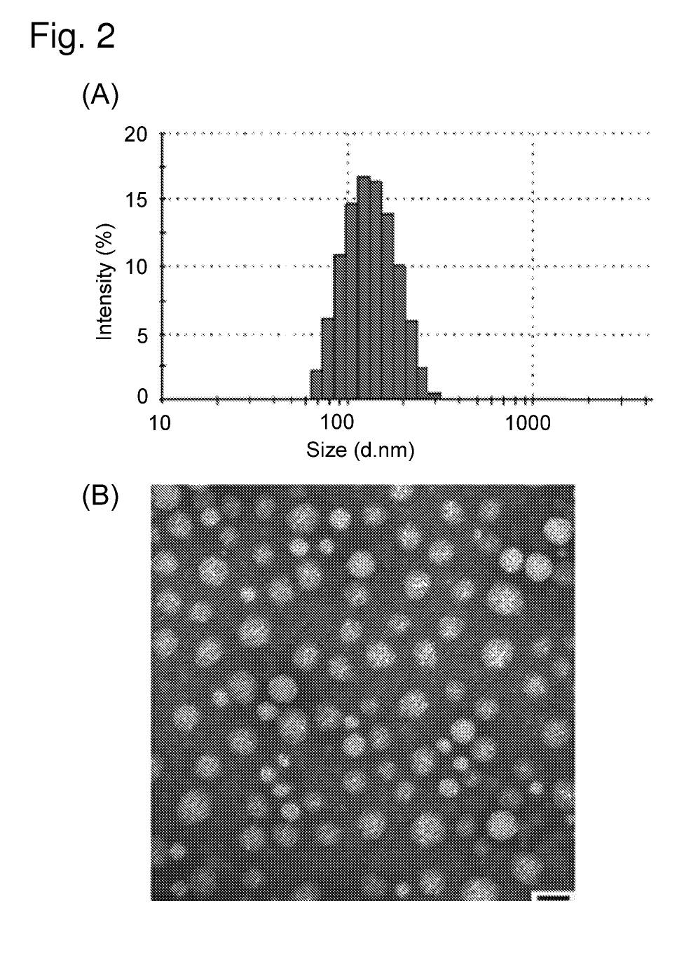

The typical DLS result indicated a uniform and narrow size distribution of the PAE micelles (FIG. 2(A)), and TEM micrograph results (FIG. 2(B)) showed a spherical morphology and moderate size distribution of micelles. As summarized in Table 2, the diameters of micelles were 115 nm for reducible rPAE and 127 nm for non-reducible nPAE. The sizes depended on the molecular weights of the copolymers. It can be explained that more PEG side chains results in the larger sized micelles due to the formation of the thicker hydration layers.

Twenty μl of pyrene acetone solution (20 μg/ml) were added to 4-ml screw vials, and then the acetone was dried under an airflow. Four ml of aqueous solution containing 2 mg/ml of PAE were added to the vials. The final concentration of pyrene in each vial was 0.1 μg/ml. After being equilibrated for 1 h at room temperature, the excitation spectra (300-360 nm) of the solutions were recorded at an emission wavelength of 395 nm with the excitation and emission bandwidths set at 5 nm.

For the reduction sensitivity assay, amounts of DTT stock solution were added into the cuvettes to produce final DTT concentrations of 0.1, 1, 2.5 or 5 mM. Then, the excitation spectra of the solutions were recorded at predetermined times. The ratios of the peak intensities at 338 nm and 334 nm (I338/I334) of the excitation spectra were recorded and plotted versus time.

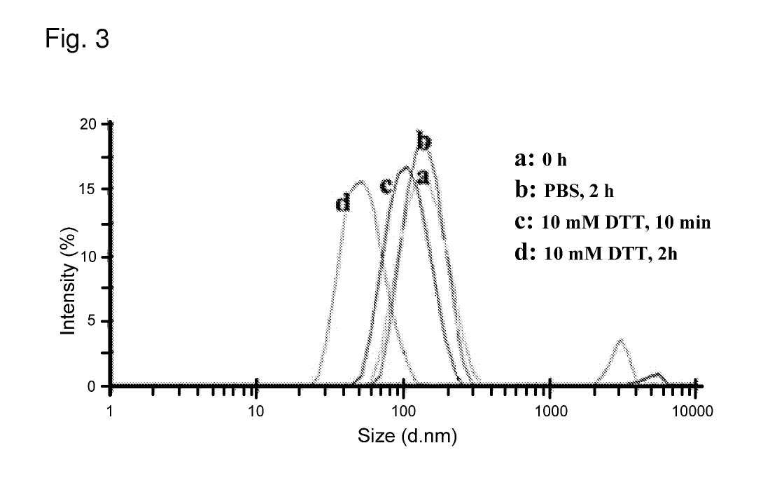

The size change of micelles in response to 10 mM DTT in PBS buffer (pH 7.4, 10 mM) was analyzed by DLS measurement. Remarkably, the size reduction and aggregation of the reducible PAE (rPAE) micelles were observed simultaneously, resulting in the appearance of two peaks after adding DTT as shown in FIG. 3. The two peaks that appear in the DLS measurements after the DTT addition are most likely due to the reductive degradation of the core of the micelles that subsequently formed smaller micellar nanoparticles and loose micellar aggregation. In contrast, no change in micelle sizes was observed after 2 h in the absence of DTT. Meanwhile, non-reducible PAE polymeric micelles maintained the size and size distribution in the presence of 10 mM DTT.

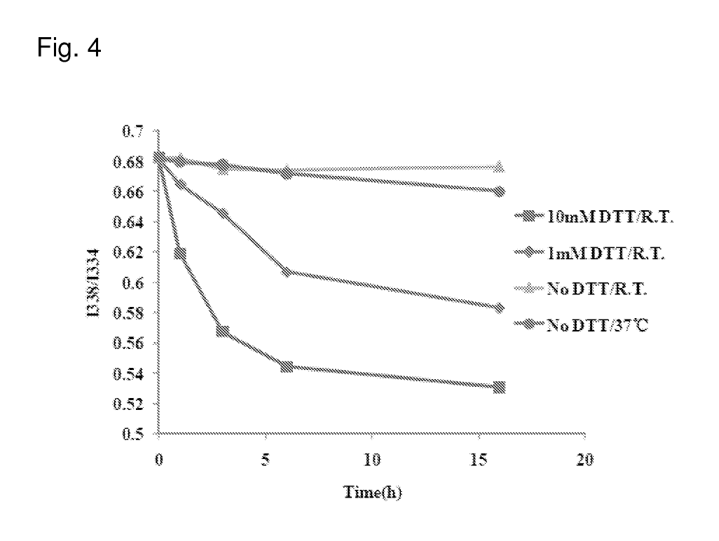

The demicellization behaviour of rPAE micelles in response to DTT was also investigated by a fluorescence spectroscopy using pyrene as the probe. As shown in FIG. 4, the ratio of I338/I334 decreased from 0.68 to about 0.58 after exposure to 1 mM DTT for 16 h. When the DTT concentrations were increased to 10 mM, the I338/I334 ratio dropped to about 0.53 in 16 h. The change of the I338/I334 ratio indicated that the surrounding environment of entrapped pyrene changed from hydrophobic core to aqueous phase, which was used to characterize the disassembly of the micelles. It appeared that the demicellization was very fast in the higher reducing reagent concentration analogous to cytoplasm. Without DTT, the I338/I334 value did not change in 16 h at room temperature and showed a very small decrease at 37° C. Combining the DLS data above, it can be concluded that the micelles can stably exist in normal physiological condition but rapidly disassemble in reducing environments such as intracellular compartments of a cell.

### Example 3

### Loading and In Vitro Release of Doxorubicin from Reducible Self-Assembled PAE Micelles

DOX-loaded micelles of PAE copolymer were prepared as follows. DOX (1 mg) was dissolved in 1 ml DMSO, then was stirred with a 1.5 equivalent of triethylamine, followed by addition of 20 mg of PAE copolymer and stirring for another 60 min. Five ml of distilled water was added dropwise under vigorous stirring. The dispersed DOX-loaded polymeric micelles were dialyzed for 24 h (molecular weight cutoff at 7000 Da) to remove free DOX and by-products. Finally, the DOX-loaded micelles were lyophilized to give a red powder. By using UV-vis spectroscopy, the drug-loading efficiency of DOX-loaded polymeric micelles dissolved in DMSO was quantified by referring to its absorbance at 480 nm. For determination of drug-loading content, DOX-loaded micelles were dissolved in DMSO and analyzed with fluorescence spectroscopy, wherein a calibration curve was obtained with DOX/DMSO solutions of different DOX concentrations.

Drug-loading content and drug-loading efficiency were calculated according to the following equations:

Drug-loading content as a percentage=weight of loaded drug/(weight of copolymer+weight of loaded drug)×100% EQ 1

Drug-loading efficiency as a percentage=weight of loaded drug/weight of feeding drug×100% EQ 2

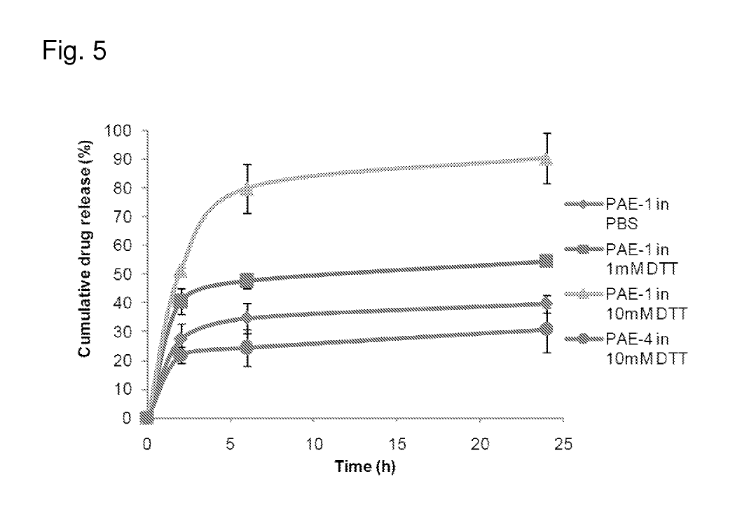

The results in FIG. 5 show that the drug-loading efficiencies were approximately 60% for the polymeric micelles. Such high drug-loading efficiencies could be due to the interaction of doxorubicin with phenyl in the copolymers via π-π interaction. Dynamic light scattering (DLS) measurements determined that the average hydrodynamic diameter of the blank PAE micelles in an aqueous solution was about 130 nm, while the size of the DOX-loaded PAE micelles was about 135 nm (Table 2). The slightly increased average size of the DOX-loaded copolymeric micelles, as compared to the blank micelles, may have been caused by the drug molecules that got entrapped in the hydrophobic rPAE cores. These results indicate that the copolymeric micelles were well-dispersed in the aqueous medium and demonstrated homogeneous nano-sized micelle structures.

The in vitro doxorubicin release behaviour of the DOX-loaded micelles was evaluated via dialysis against PBS buffer (pH=7.4, 0.1 M) in the presence and absence of DTT. The DOX release measurements were conducted as follows. Dispersed DOX-loaded polymeric micelles were added to a dialysis membrane tube (molecular weight cutoff at 7000 Da), and the incubation solutions were 30 ml PBS (pH 7.4) with DTT concentrations of 0.01, 1 or 5 mM. At predetermined frequencies, 6 ml of incubated solution were taken out, and 6 ml of fresh PBS were added to refill the incubation solution to 30 ml. DOX-release profiles were determined by measuring the UV-vis absorbance of the solutions at 480 nm. DTT solutions of 1 and 10 mM concentrations were chosen to mimic the reducing-agent level in different subcellular compartments.

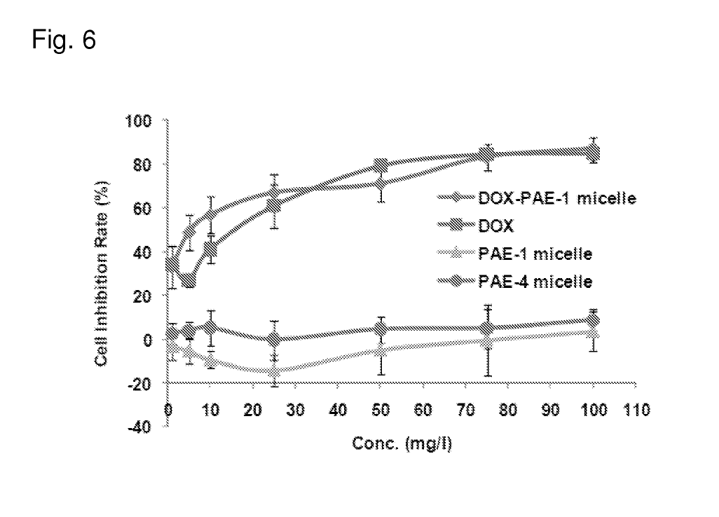

As shown in FIG. 5, rPAE micelles released about 30% of their sequestered DOX in the absence of DTT in a 24-h period. In the presence of 1 mM DTT, the DOX-loaded rPAE micelles released about 50% of their sequestered DOX within a 24-h period. When the DTT concentration was increased to 10 mM, a faster release of DOX from the micelles was detected, with about 80% of their sequestered DOX was released in the first 6 h and up to 90% of the sequestered DOX was released in 24 h. In contrast, the DOX-loaded non-reducible rPAE polymeric micelles exhibited minimal drug release (˜20%) in 24 h in the presence of 10 mM DTT (FIG. 6). This DTT-dependent drug release behaviour of DOX-loaded micelles is in agreement with the reduction-sensitivity characterization of PAE copolymer micelles shown in FIG. 4. High DTT concentration leads to a rapid reductive degradation of the PAE core, followed by a triggered rapid release of DOX into the surrounding solution. This reduction-induced drug release behaviour facilitates intracellular drug delivery. It is known that glutathione (GSH) is the most abundant intracellular thiol source present in milli-molar concentrations inside the cell but only occurs in micro-molar concentrations in the blood. It is therefore desirable to hypothesize that modified reduction sensitive micelles disclosed herein can retain a large portion of the drug they contain after intravenous administration into the patient and stay in the circulation at normal physiological conditions (pH 7.4, very low level of reducing reagent). Therefore, the present reductively degradable micelles have an increased opportunity to aggregate around tumours. Once the micelles reach the tumor site by way of EPR effect and are internalized inside the cells by endocytosis, faster release due to micelle disassembly might occur inside the endosome/lysosome of tumour cells because of the high GSH concentration. Therefore, this reduction-sensitive micelle may offer a potent approach to achieve efficient intracellular drug delivery.

### Example 4

### Cytotoxicity of DOX-Loaded Reducible Self-Assembled PAE Micelles

Hepatoma cells (HepG2) were used to investigate cell inhibition of DOX-loaded nanoparticles. HepG2 cells were obtained from the American Type Culture Collection and cultured with Dulbecco's Modified Eagle's Medium (DMEM, GIBCO) supplemented with 10% fetal bovine serum (FBS, GIBCO), 1.0×10⁵ U/l penicillin (Sigma) and 100 mg/l streptomycin (Sigma) at 37° C. in 5% CO2.

The cytotoxicity of the copolymeric micelles loaded with DOX was determined using the MTT Cell Proliferation Kit (Biotium Inc., Hayward, Calif., USA). HepG2 cells were seeded into a 96-well tissue culture plate at a density of 8,000 cells per well and were incubated at 37° C. in 5% CO₂. The growth medium was replaced with fresh DMEM after 24 h. Then the DOX-loaded PAE polymeric micelle solution and controls (free DOX and blank micelle solutions) were added into wells (six wells per sample). After 48 h of incubation, 10 μL MTT solution was added to each well and incubation continued for another 4 h. The medium was removed and 200 μL DMSO was added into each well to dissolve the formazan by pipetting in and out several times. The absorbance of each well was measured using an ELISA plate reader at a test wavelength of 570 nm and a reference wavelength of 630 nm. The cell inhibition of samples was calculated as:

$$Cell inhibition ( \% ) = \frac{{I}_{control} - {I}_{sample}}{{I}_{control}} \times 100 \% \qquad EQ 3$$

where Iₛₐₘₚₗₑ and I_{control} represent the intensity determined for cells treated with different samples and for control cells (untreated), respectively.

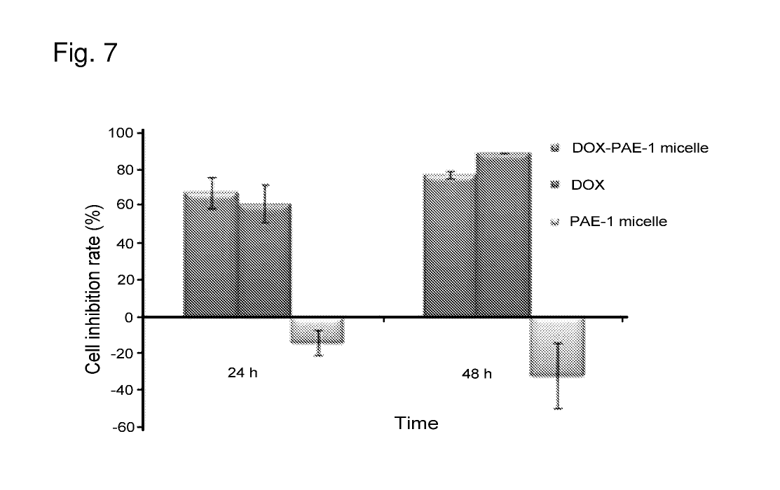

As shown in FIG. 6, empty reducible rPAE micelles showed no cytotoxicity in the concentration lower than 100 mg/L, while non-reducible nPAE micelles showed very low cytotoxicity as well but higher cytotoxicity compared to rPAE micelles. Factors that can contribute to the polymer cytotoxicity include molecular weight, chemical architecture and charge density. Herein, two polymers have similar chemical architecture and charge density, while they differ in their disulfide bonds content and subsequently in their degradability. This relationship of disulfide bond content and polymer cytotoxicity indicates that the degradation of reducible polymer in the reductive intracellular environment contributes to the observed decrease in cytotoxicity. As expected, the cytotoxicity of DOX increases with increasing dose. Notably, the cytotoxicity potential of the DOX-loaded micelles was identical to the free doxorubicin in 24 h and 48 h (FIG. 7). Since DOX is a small molecule drug, it can easily diffuse into cytoplasm and there exert its function. In contrast, the DOX-loaded micelles are most likely internalized by endocytosis. The IC50 (the dose having 50% cell inhibition) of nanoparticle-encapsulated DOX was about two times lower than free doxorubicin. The data disclosed here indicate that the nano-scale carrier is safe and can efficiently deliver doxorubicin into the cytosol of human cells and thereby boost its cell-killing effect.

### Example 5

### Cellular Uptake of DOX-Loaded Reducible Self-Assembled PAE Micelles by Hepatocellular Carcinomatous HepG2 Cells

Confocal laser scanning microscopy (CLSM) was employed to examine the cellular uptake of DOX by incubating hepatocellular carcinomatous HepG2 cells with free DOX or the DOX-loaded micelles for 15 min, 2 h or 24 h. First, the HepG2 cells were seeded in culture dishes at a density of 2×10⁵ cells/dish, overlaid with cover slips, and cultured for 24 h. Then, the cells were exposed to DOX-loaded micelles. After predetermined incubation times, the cover slips were removed and was washed with cold PBS three times after which, the cells were fixed by 4% paraformaldehyde in PBS at room temperature for 15 min. After fixation, the cells were permeabilized by 0.1% Triton X-100 in PBS for 10 min and then rinsed with PBS three times. The cells were incubated in 10 uM BODIPY® FL phallacidin/1% (w/v) BSA solution for 20 min and then rinsed with PBS three times (BODIPY is a registered trademark of Molecular Probes Inc., Eugene, Oreg., USA). The cells were then incubated in 10 μM Topro-3 for 20 min and then rinsed with PBS three times. The cover slips were set onto microscope slides and examined by CLSM.

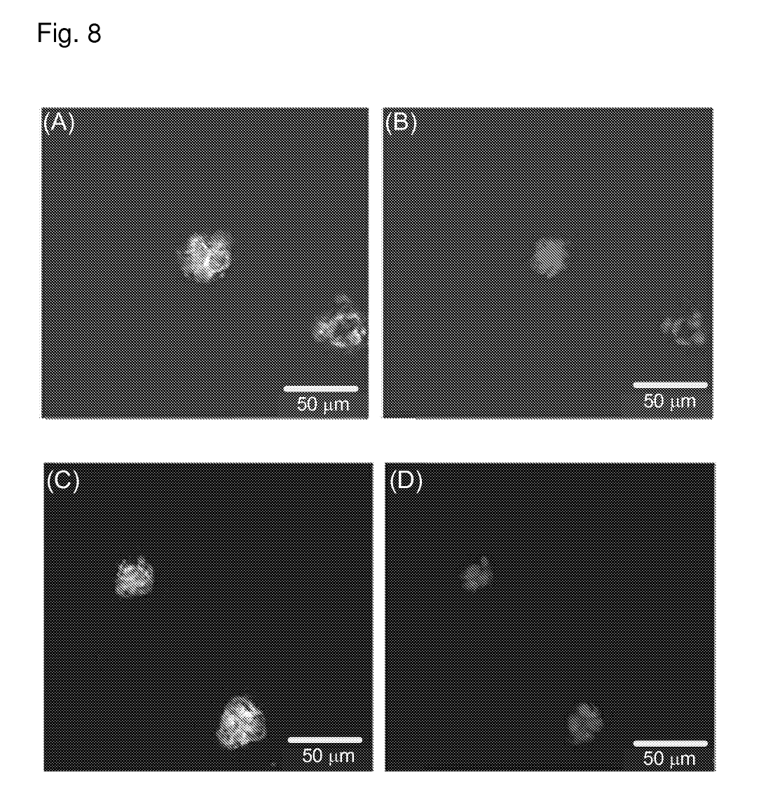

As shown in FIGS. 8(A) and 8(B), the free DOX was visibly observed and evenly distributed in cell cytoplasm and the nuclei of the HepG2 cells after 15-min incubation. Remarkably, FIGS. 8(C) and 8(D) showed DOX fluorescence in the cytoplasm after only 15-min of incubation with the DOX-loaded rPAE micelles. It has previously been shown that cell membranes are naturally impermeable to nanoparticles that have molecular weights larger than 1 kDa (e.g., Mukherjee et al., 1997, Endocytosis. Physiol. Rev. 77:759-803; Bareford et al., 2007, Endocytic mechanisms for targeted drug delivery. Adv. Drug Del. Rev. 59:748-758). While the molecular weight of free DOX is 543.52 Da, the average weight of a reducible self-assembled PAE micelle is over 10 kDa. Therefore, the free DOX molecule could be internalized into tumor cells with molecular diffusion mechanism while DOX-loaded PAE micelles most likely need to be endocytosed, which is an efficient route for drug entry through cell membranes.

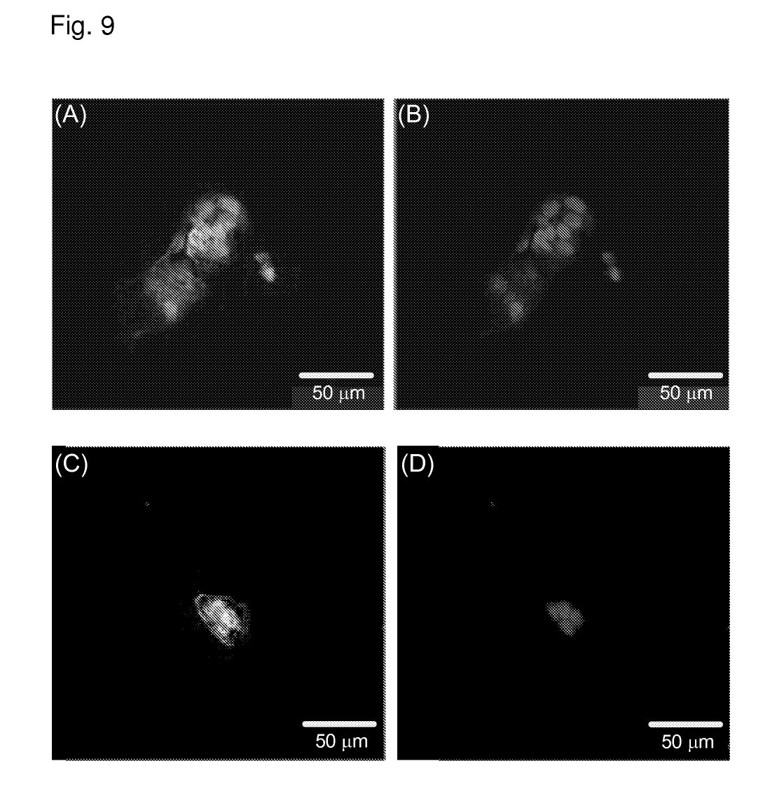

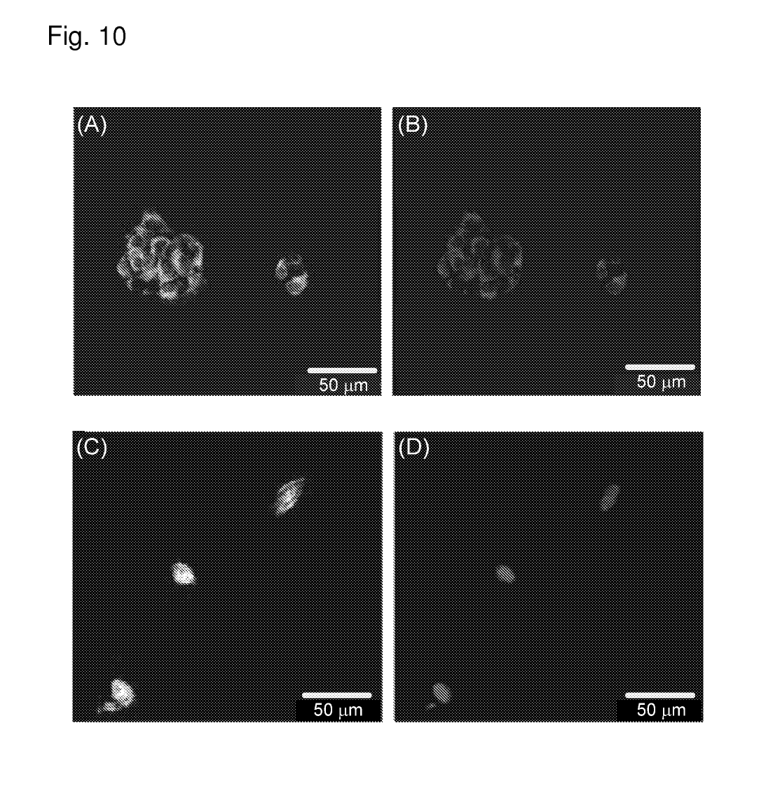

Interestingly, after 2 h exposure to free DOX or DOX-loaded micelles, DOX fluorescence accumulation was detected in and around the nuclei (FIGS. 9(a)-9(D)). DOX acts through interaction with DNA by intercalation and inhibition of macromolecular biosynthesis. After 24 h exposure to free DOX and DOX-loaded micelles, most of DOX fluorescence was distributed into the nucleus of the HepG2 cells (FIGS. 10(A)-10(D)). However, the nucleus became distinctively swollen and the cytoplasm shrank after being exposed to DOX and DOX-loaded micelles for 24 h in comparison to cells that were exposed to these agents for shorter time periods. This observation further supports the effective and efficient delivery of DOX into the cells by the reducible self-assembled micelles disclosed herein which increased the DOX entry into nucleus.

Combined with the determination of reduction sensitivity of micelles as shown in FIG. 4, and the in vitro drug release as shown in FIG. 5, the cell uptake process can be deduced that the micelles with DOX were endocytosed thus forming endosomes, and the polycationic nature of micelles facilitated endosomal escape followed by the collapse of micelles in exposure to the high level of reductants in cytoplamic environment thus release of the entrapped DOX. Considering the reductant level in cytoplasm is in range of 1-10 mM, it can be predicted that the drug-encapsulating reducible self-assembled micelles would undergo the fast disassembly as shown in FIG. 4, with concurrent fast drug release as shown in FIG. 5.

### Example 6

### Cellular Uptake of DOX-Loaded Reducible Self-Assembled PAE Micelles by MCF-7 Human Breast Cancer Cells

Confocal laser scanning microscopy (CLSM) was employed to examine the cellular uptake of DOX by incubating MCF-7 human breast cancer cells with free DOX or DOX-loaded reducible self-assembled rPAE micelles or DOX-loaded non-reducible self-assembled nPAE micelles for 15 min and 2 h. First, the MCF-7 cells were seeded in 10-cm culture dishes at a density of 2×10⁵ cells/dish, overlaid with cover slips, and cultured for 24 h. Then, the cells were exposed to DOX-loaded micelles. After the predetermined incubation times, the cover slips were removed and washed with cold PBS three times after which, the cells were fixed by 4% paraformaldehyde in PBS at room temperature for 15 min. After fixation, the cells were permeabilized by 0.1% Triton X-100 in PBS for 10 min and then rinsed with PBS three times. The cells were incubated in 10 uM BODIPY® FL phallacidin/1% (w/v) BSA solution for 20 min and then rinsed with PBS three times. The cells were then incubated in 10 μM Topro-3 for 20 min and then rinsed with PBS three times. The cover slips were set onto microscope slides and examined by CLSM.

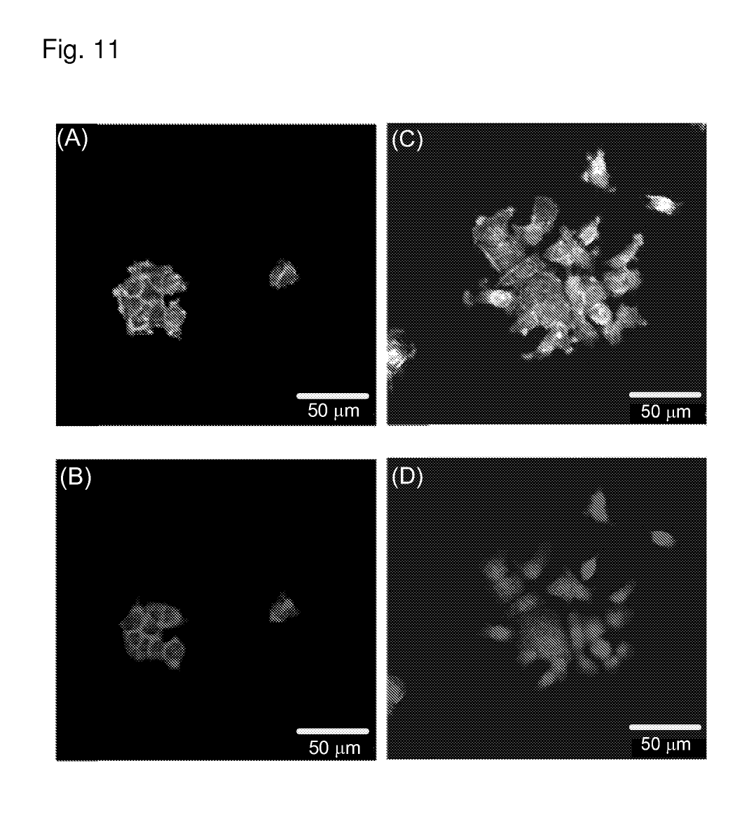

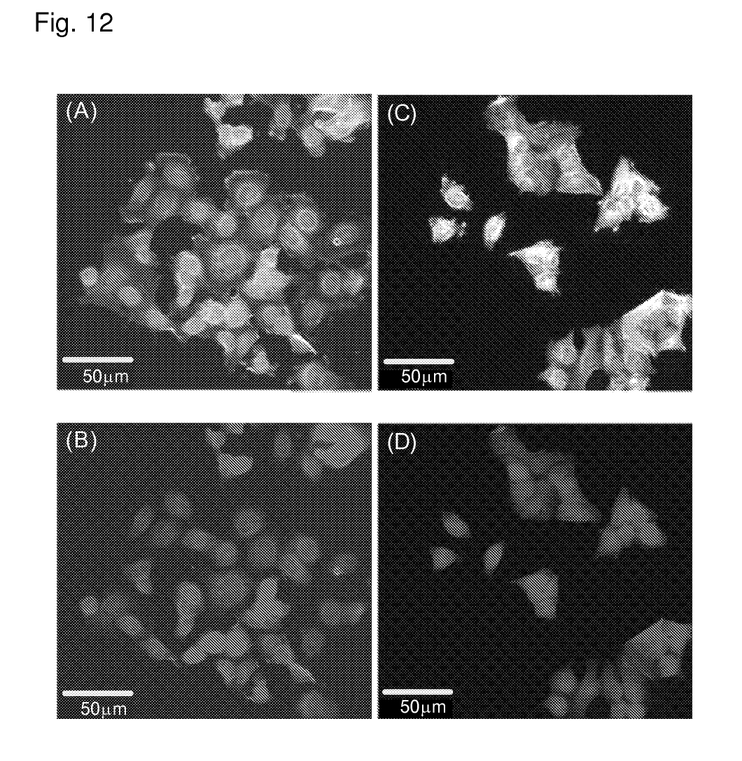

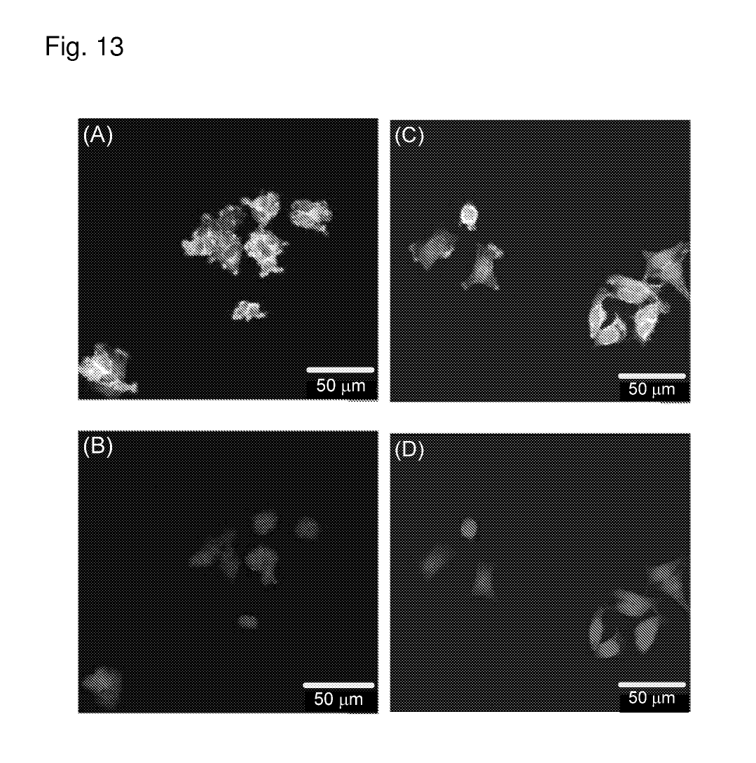

As shown in FIG. 6, After 15 min of incubation of the MCF-7 cancer cells in all three preparations, i.e., the free DOX, DOX-rPAE micelles, DOX-nPAE micelles, fluorescing DOX molecules were observed intracellularly in all three formulations (FIGS. 11, 12, 13, respectively). After 2 hr of incubation, accumulations of DOX molecules around the nuclei of MCF-7 cancer cells were observed in all three treatments. This indicated that the two kinds of copolymeric micelles could rapidly and efficiently deliver the loading cargos to cytoplasm, which may be attributed to the poly(β-amino ester) backbone and their cationic nature. The reductively degradable PAE micelles could enhance the cytoplasmic DOX delivery and therefore nucleus localization of DOX, which would facilitate the anti-tumour efficacy of DOX.

In summary, a safe and efficient drug delivery system based on poly(β-amino ester)-g-poly(ethylene glycol) amphiphilic copolymers is disclosed. After the nano-sized micellar nanoparticles have self-assembled in aqueous solution, the micelles can entrap DOX within their core-shell structures with high-loading efficiency. DOX-loaded micelles are stable in normal physiological conditions. However, the encapsulated drug will be quickly released in a high-level DTT conditions which will lead to fast disassembly of micelles. The MTT assay results indicate the safety of this series of polymeric micelles and show comparative cell-killing efficiency as that of doxorubicin. Remarkably, the reducible micelles showed lower cytotoxicity and faster internalization in comparison to the non-reducible micelles.

1. A reductively degradable micelle for sequestering therein an anthracycline, the micelle comprising:
a plurality of poly(β-amino ester)-graft-poly(ethylene glycol) amphiphilic copolymers; and
a plurality of phenylbutylamine functional side groups attached to the plurality of poly(β-amino ester)-graft-poly(ethylene glycol) amphiphilic copolymers.

2. The reductively degradable micelle of claim 1, wherein the phenylbutylamine functional side groups are butylbenzene chains.

3. The reductively degradable micelle of claim 1, wherein the anthracycline is doxorubicin.

4. A reductively degradable micelle for sequestering therein an anthracycline, wherein the reductively degradable micelle is of the formula: 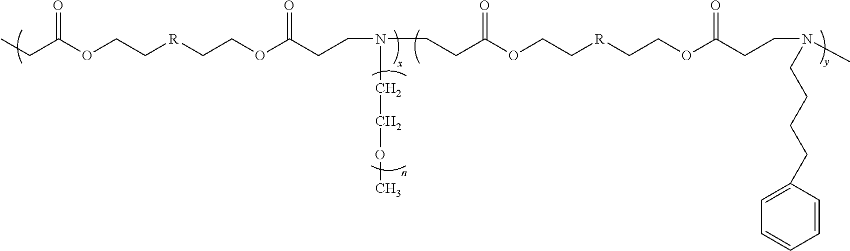
wherein:
R═S—S, or CH₂—CH₂.

5. The reductively degradable micelle of claim 5, wherein the anthracycline is doxorubicin.

6. Anthracycline-loaded reductively degradable micelles, the micelle comprising:
a plurality of poly(β-amino ester)-graft-poly(ethylene glycol) amphiphilic copolymers;
a plurality of phenylbutylamine functional side groups attached thereto the plurality of poly(β-amino ester)-graft-poly(ethylene glycol) amphiphilic copolymers; and
an anthracycline sequestered therein the micelles.

7. The anthracycline-loaded reductively degradable micelles of claim 6, wherein the phenylbutylamine functional side groups are butylbenzene chains.

8. The anthracycline-loaded reductively degradable micelles of claim 6, wherein the anthracycline is doxorubicin.

9. Anthracycline-loaded reductively degradable micelles for sequestering therein an anthracycline, wherein the reductively degradable micelles are of the formula: 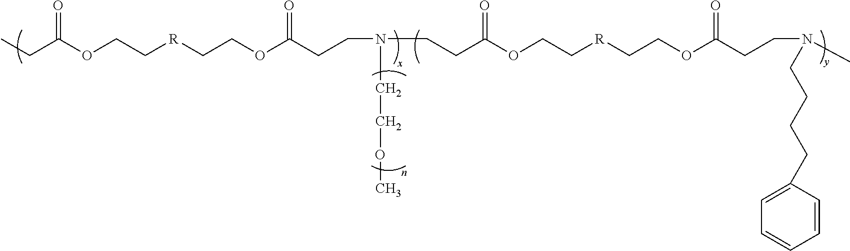
wherein:
R═S—S, or CH, —CH₂.

10. The anthracycline-loaded reductively degradable micelles of claim 9, wherein the anthracycline is doxorubicin.

11. A method for producing reductively degradable micelles suitable for sequestering therein an anthracycline, the method comprising the steps of:
dissolving 2′-dithiodiethanol diacrylate with 4-phenylbutylamine and methoxy-polyethyleneglycol(5K)—NH₂ in dimethyl sulfoxide to produce poly(β-amino ester)-graft-poly(ethylene glycol) amphiphilic copolymers (PAE) having phenylbutylamine side groups;
precipitating the PAE copolymers with diethyl ether;
drying the precipitated PAE copolymers;
solubilizing the PAE copolymers in fresh dimethyl sulfoxide and adding distilled water thereto; and
dialysizing the solubilized PAE copolymers against distilled water to produce reductively degradable micelles having a molecular weight cut-off of 700 Da.

12. A method for producing anthracycline-loaded reductively degradable micelles, the method comprising the steps of:
dissolving a selected anthracycline in dimethyl sulfoxide;
commingling the dissolved anthracycline with the reductively degradable micelles of claim 1 thereby loading the reductively degradable micelles with dissolved anthracycline;
dialysizing the anthracycline-loaded reductively degradable micelles; and
drying the dialysized anthracycline-loaded reductively degradable micelles.

13. The method of claim 12, wherein the anthracycline is doxorubicin.

14. A composition comprising a plurality of reductively degradable micelles sequestering therein an anthracycline, and a pharmaceutically acceptable carrier therefore, the reductively degradable micelles comprising:
a plurality of poly(β-amino ester)-graft-poly(ethylene glycol) amphiphilic copolymers;
a plurality of phenylbutylamine functional side groups attached thereto the plurality of poly(β-amino ester)-graft-poly(ethylene glycol) amphiphilic copolymers; and
an anthracycline sequestered therein the micelles.

15. The composition of claim 14, wherein the anthracycline is doxorubicin.
